(19) United States
(12) Patent Application Publication
(10) Pub. No.: US 2008/0090918 A1
(43) Pub. Date: Apr. 17, 2008

(54) Separation medium, its preparation and its use

(75) Inventors: Bo Mattiasson, Hjarup (SE); Igor Galaev, Lund (SE); Vladimir Lozinsky, Moscow (RU); Fatima Plieva, Lund (SE)

Correspondence Address: MCDERMOTT WILL & EMERY LLP, 600 13TH STREET, N.W., WASHINGTON, DC, 20005-3096, US

(73) Assignee: PROTISTA BIOTECHNOLOGY AB, Bjuv (SE)

(21) Appl. No.: 12/000,042

(22) Filed: Dec. 7, 2007

Related U.S. Application Data
(62) Division of application No. 10/492,429, filed on Aug. 17, 2004.

(30) Foreign Application Priority Data
Oct. 12, 2001 (SE) 0103403-2

(51) Int. Cl. B01D 39/16; C12N 7/02

(52) U.S. Cl. 516/106; 435/239; 516/105

(57) ABSTRACT

A separation medium in macroporous gel form is disclosed which is obtainable by cooling an aqueous solution of at least one gel forming polymer to a temperature, at which the solvent in the system is partially frozen with the dissolved substances concentrated in the non-frozen fraction of the solvent, said gel forming polymer being selected from the group consisting of polymers normally forming gels too fast when an aqueous solution thereof is cooled to a temperature within a range below 0° C. to enable the formation of a cryogel and said cooling being carried out in the presence of at least one chaotropic agent in said aqueous solution in order to prevent gel formation before the polymer solution is frozen. The use of said separation medium for diverse separation purposes is also disclosed.

### TECHNICAL FIELD

The present invention relates to a separation medium, its preparation and its use. More particularly, the invention relates to a separation medium in macroporous gel form, its preparation by cooling an aqueous solution of a gel forming polymer to a temperature, at which the solvent in the system is partially frozen with the dissolved substances concentrated in the non-frozen fraction of the solvent, in order to form a cryogel and the use of said separation medium.

### BACKGROUND ART

Recent progress in biosciences resulted in redirecting of research interests to a large extent from individual biomolecules to the problems how these biomolecules are organized in more complex structures and how these structures function in the living cell. Extensive experience of working with individual biomolecules resulted in the development of numerous highly efficient techniques for the isolation and purification of molecular objects with molecular weights less than 10⁶ Da. Contrary, the purification of larger objects, often combined under the name of nanoparticles, like plasmids, cell organelles, viruses, protein inclusion bodies, macromolecular assemblies as well as the separation of cells of different kind still remains a challenge. Large particle sizes (100-1000 nm), low diffusion rates, and complex molecular surfaces distinguish such objects from protein macromolecules (commonly <10 nm).

Traditionally used approaches for isolation of nanoparticles, as ultracentrifugation and micro/ultrafiltration are limited either in scale or resolution due to the similarities of size and density of cell debris and target nanoparticles. Partitioning in aqueous two-phase systems could be used alternatively for the isolation of nanoparticles but it suffers from the necessity to separate the target product from the phase-forming polymer.

Selective adsorption to a chromatographic matrix is a method, which offers many potential advantages with respect to resolution scale-up and process integration. It is noteworthy that only a small number of commercial chromatographic matrixes such as Sephacryl S-1000 SF from Amersham Pharmacia are claimed to accommodate spherical particles up to 400 nm in diameter within the intra-particle pores.

Nanoparticles and cells have very low diffusion coefficients due to the large size and they could be forced inside the pores only by a convective flow. For beaded chromatographic matrices most of the convective flow in the column goes through the voids in between the beads. Even for recently developed superporous beads with pore size of 800 nm up to 95% of the flow goes through the voids around the beads.

In early 90-s Svec, F. and Fréchet, J. M., Science 273:205-211 (1996), suggested to use molded continuous chromatographic media or so called macroporous monoliths, produced by the controlled polymerization inside the chromatographic column. Typically these monoliths are produced by polymerization of styrene or acrylate monomers and contain flow-through pores with diameters in the range of 700-2000 nm (0.7-2 μm). Later on, continuous superporous chromatographic media with pores as large as 20-200 μm were produced from agarose by Gustavsson, P. E. and Larsson, P-O., J. Chromatog. A. 795:199-210 (1998); Braas, GMF, et al., Trans. Inst. Chem. Eng. 78:11-15 (2000). These pores could easily accommodate objects as large as yeast cells.

Cryogels have appeared recently as a new class of materials with a combination of unique properties. Highly porous polymeric materials with a broad variety of morphologies could be produced from practically any gel-forming precursors using cryotropic gelation technique.

Cryotropic gelation (cryogelation or cryostructuration are often used synonyms) is a specific type of gel-formation which takes place as a result of cryogenic treatment of the systems potentially capable of gelation. The essential feature of cryogelation is compulsory crystallization of the solvent, which distinguishes cryogelation from chilling-induced gelation when the gelation takes place on decreasing temperature e.g. as gelation of gelatine or agarose solutions which proceeds without any phase transition of the solvent.

The processes of cryogelation have some unique characteristics.

1. Cryotropic gel formation is a process which proceeds in a non-frozen liquid microphase existing in the macroscopically frozen sample. At moderate temperatures below the freezing point some of the liquid remains still non-frozen accumulating in high concentrations (so called cryoconcentrating) all the solutes present in the initial solution. Chemical reactions or processes of physical gelation proceed in the non-frozen microphase at apparently much higher concentrations than in the initial.

2. The result of cryoconcentrating of dissolved substances in non-frozen liquid is a decrease in the critical concentration of gelation as compared to traditional gelation at temperatures above the freezing point.

3. Usually cryogelation in moderately frozen samples proceeds faster than traditional gelation at temperatures above the freezing point.

4. Frozen crystals of the solvent play a role of porogen when cryogels are formed producing a system of interconnected macropores. The macropore size could be as large as a few hundreds μm (Ø). The cryogels have often sponge-like morphology contrary to continuous monophase traditional gels produced from the same precursors at temperatures above freezing. Most of the solvent in cryogels is capillary bound and could be easily removed mechanically.

5. Temperature dependence of cryogelation has usually an optimum due to the balance between the effects facilitating gelation (cryoconcentrating) and factors decelerating it (low temperature, high viscosity in liquid microphase).

6. Cryogels are mechanically strong, but non brittle due to the elasticity of polymer walls in between macropores.

7. The porosity, mechanical strength and density of cryogels could be regulated by the temperature of cryogelation, the time a sample is kept in a frozen state and freezing/thawing rates.

The production of cryogels in general is well documented. For a review, vide e.g. Kaetsu, I., Adv. Polym. Sci. 105:81 (1993); Lozinsky, V. I. and Plieva, F. M., Enzyme Microb. Technol. 23:227-242 (1998); and Hassan, Ch. M. and Peppas, N. A., Adv. Polym. Sci. 151:37 (2000).

The most intensely studied cryogels are those prepared from poly(vinyl alcohol) (PVA) due to their easy availability. Thus when cooling an aqueous solution of PVA to a temperature within a range below 0° C. the ratio between gelling of the PVA and the crystallization of water is such that cryogels are easily formed. In comparison therewith, other polyhydric gel forming polymers, e.g. polysaccharides such as agarose, agar and carrageenans and protein based polymers such as gelatine (concentrated solutions) are forming gels too fast (or alternatively, to slow as, e.g., for the solutions of albumins) when an aqueous solution thereof is cooled to a temperature within a range below 0° C. to enable the formation of cryogels, which can be used as a macroporous separation medium.

It is an object of the present invention to provide a method by which a separation medium in macroporous gel form can be prepared from a wider range of gel forming polymers than hitherto possible by cooling an aqueous solution of the gel forming polymer to a temperature within a range below 0° C.

It is another object of the present invention to provide a method which introduces a further variable in the preparation of cryogels from gel forming polymers by which the rate of gelation can be controlled.

It is a further object of the present invention to provide a method which introduces a further variable in the preparation of cryogels which facilitates the tailoring of the properties of cryogels made from gel forming polymers.

It is still another object of the invention to provide a new separation medium in macroporous gel form, especially a separation medium based on gel forming polymers which could not effectively be used previously for the preparation of cryogels.

These and other objects are attained by means of the present invention.

### DISCLOSURE OF THE INVENTION

The present invention is based on the finding that the rate at which a gel is formed when cooling an aqueous solution of a gel forming polymer to a temperature at which the solvent in the system is partially frozen with the dissolved substances concentrated in the non-frozen fraction of the solvent, can be lowered in a controlled way by adding a chaotropic agent to said aqueous solution, which without additions forms gels too fast when cooled down to enable the formation of macroporous cryogels, and that such an addition enables the preparation of macroporous gel useful as separation media. The aqueous solution may consist of water as solvent or a mixture of water and a water-miscible organic solvent.

On basis of this finding the present invention provides according to one aspect thereof a separation medium in macroporous gel form obtainable by cooling an aqueous solution of at least one gel forming polymer to a temperature, at which the solvent in the system is partially frozen with the dissolved substances concentrated in the non-frozen fraction of the solvent, said gel forming polymer being selected from the group consisting of polymers normally forming gels too fast when an aqueous solution thereof is cooled to a temperature within a range below 0° C. to enable the formation of a cryogel and said cooling being carried out in the presence of at least one chaotropic agent in said aqueous solution in order to prevent gel formation before the polymer solution is frozen.

According to another aspect of the present invention there is provided a method for the preparation of a separation medium in macroporous gel form by cooling an aqueous solution of at least one gel forming polymer to a temperature, at which the solvent in the system is partially frozen with the dissolved substances concentrated in the non-frozen fraction of the solvent, which method is characterized in that said gel forming polymer is selected from the group consisting of polymers normally forming gels too fast when an aqueous solution thereof is cooled to a temperature within a range below 0° C. to enable the formation of a cryogel and that said cooling is carried out in the presence of at least one chaotropic agent in said aqueous solution in order to prevent gel formation before the polymer solution is frozen.

Examples of gel forming polymers to be used in the present invention are polysaccharides selected from the group consisting of agarose, agar, carrageenans, starch and cellulose and their respective derivates and mixtures of said polysaccharides.

The gel forming polymers can be used alone or as a mixture of two or more thereof. A mixture of a gel forming polymer and another not gel forming polymer, e.g. a polymer acting as a cross-linking agent, may also be contemplated.

According to the present invention cooling of the aqueous solution of said at least one gel forming polymer is carried out in the presence of at least one chaotropic agent. Preferably said at least one chaotropic agent is selected from the group consisting of urea, alkyl ureas, guanidine chloride, LiCl, KSCN, NaSCN, acids and bases and mixtures thereof.

As acids and bases inorganic acids and bases as well as organic acids and bases can be used. Examples of acids and bases contemplated for use in the present invention are hydrochloric acid, hydrobromic acid, hydroiodic acid, perchloric acid, trifluoro acetic acid, sulfuric acid, nitric acid, phosphoric acid, alkyl and aryl sulfonic acids, alkyl and aryl phosphonic acids, sodium hydroxide, potassium hydroxide and lithium hydroxide. These acids and bases are generally held to be strong acids and bases. However, weaker acids such as acetic acid and bases such as ammonia are also contemplated for use in the present invention although requiring more thereof to be added.

Usually, the chaotropic agent will be added to the aqueous solution to a concentration within the range of from 0.01 M to 5 M in the solution. However, as is readily understood by a man of ordinary skill in the art, the optimum concentration to be used in each specific case will to a decisive extent depend upon such factors as the specific polymer or polymers and chaotropic agent or agents used, the concentration of the polymer or polymers, the rate of gel formation wanted, the temperature of cooling and so on.

Strong acids and bases as represented by hydrochloric acid and sodium hydroxide, are generally used at a concentration within the range of from 0.01 M to 0.3 M, weak acids, such as acetic acid may be used at a concentration of from 0.5 M to 1.5 M, whereas e.g. urea and KSCN are used at a concentration of from 1 to 5 M.

The chaotropic agent is preferably added to the solution of the gel forming polymer but may also be added to the water before the gel forming polymer is added or to a dispersion of the gel forming polymer in water before or during the dissolution of said dispersion to dissolve said polymer.

Chilling or cooling of the solution of the gel forming polymer or polymers and chaotropic agent or agents is generally carried out to a temperature within the range of from −5° C. to −40° C., preferably from −10° C. to −30° C. Water present in the solution is partially frozen at these temperatures with the dissolved substances concentrated in the non-frozen fraction of water. As is generally perceived by the man of ordinary skill in the art of cryogel preparation the optimum temperature will vary depending on the concentrations of the polymer(s) and chaotropic agent(s) in solution in the specific case and the target properties of the cryogel such as the pore size, thickness of walls in between pores and the mechanical strength of the gel.

According to an embodiment of the separation medium of the present invention said polymer is in a cross-linked form. Cross-linking is generally carried out after the formation of the cryogel but cross-linking during cryogel formation is also possible.

Cross-linking may be achieved by means of cross-linking agents generally known in the art of cross-linking polymers contemplated for use in the present invention. Thus the polymer may, for instance, be cross-linked by means of a crosslinking agent selected from the group consisting of epichlorohydrin, divinyl sulfone, glutaric dialdehyde, di- and triglycidyl compounds, such as, for instance, diglycidyl-1,2-cyclohexane dicarboxylate, diglycidyl-1,2,3,6-tetrahydrophtalate, N,N-diglycidylaniline, and N,N-diglycidyl-4-glycidyloxyaniline, azidobenzoyl hydrazide, 4-(N-maleimidomethyl)cyclohexane-1-carboxyl hydrazide hydrochloride, N-hydroxysuccinimidyl-4-azidosalicylic acid, 3-(2-pyridyldithio)-propionyl hydrazide, dimethyladipimidate.2HCl, N-succinimidyl-6(4′-azido-2′-nitrophenylamino)hexanoate and sulfosuccinimidyl-(4′-azidosalicylamido) hexanoate.

According to another embodiment of the separation medium of the present invention said separation medium has been modified by introducing a member selected from the group consisting of ligands, charged groups and hydrophobic groups thereinto.

The ligand to be introduced into the separation medium according to the invention can be varied within wide limits. Preferably, the ligand is selected from the group consisting of peptides, metal chelates, sugar derivatives, boronate derivatives, enzyme substrates and their analogues, enzyme inhibitors and their analogues, protein inhibitors, lectins, antibodies and their fragments and thiol-containing substances. The ligands are attached to the separation medium via at least one covalent bond between the ligand and the separation medium. Particulate structures may represent ligand activity which also can be utilized in the proposed cryogels. These particulate structures do not need to be covalently bound. Alternatively, reversible immobilization e.g. via electrostatic interactions can be used for the immobilization of the desired ligand.

According to a further embodiment of the separation medium of the present invention said separation medium has become modified by introducing a member selected form the group consisting of dyes e.g. Cibacron Blue 3 GA covalently coupled to OH- or NH₂-carrying separation medium via triazine group and ion exchange groups e.g. dimethylaminoethyl group covalently coupled to the separation media containing epoxy groups, thereinto.

According to a still further embodiment of the separation medium of the present invention a filler is present in the separation medium in order to increase the density thereof to introduce a ligand thereinto.

A filler to be used according to this embodiment of the present invention may be selected from the group consisting of metals and metal oxides, such as titanium dioxide, molybdenum powder, zirconium dioxide iron oxide, stainless steel powder, and ion exchange substances in the form of particles.

The separation medium according to this embodiment of the invention is prepared by carrying out the cooling and partial freezing of the aqueous solution of gel forming polymer(s) and chaotropic agent(s) in the presence in said solution of said filler.

The filler may be used in an amount of from 0 to 50% by weight calculated on the total weight of the filled cryogel formed, preferably from 5 to 20% by weight.

The separation medium according to the present invention may be in the form of a monolith encased in a column. In this case cooling and partial freezing of the solution of the gel forming polymer to the formation of a cryogel is carried out with said solution within the column.

According to this embodiment the gel forming polymer is suspended in water or an aqueous solution of chaotropic agent(s) and heated, if necessary, with stirring until the complete dissolution of the polymer. Then chaotropic agent(s), if not already present in sufficient amount, is/are added and the solution is poured into the column. The content of the column is then cooled inside the column at a predetermined temperature, at which water in the system is partially frozen with the dissolved substances concentrated in the non-frozen fraction of water and for a predetermined time whereafter it is thawed. The column is rinsed with water to wash out soluble fractions.

Alternatively, the separation medium according to the invention is prepared in the form of particles. The preparation of cryogels in particle form has been extensively described in literature. V. I. Lozinsky, Zubov A. L., The plant for formation of spherical granules from material based on aqueous systems, Russian Federation Patent 2036095 (20.10.1992).

In short, an aqueous solution of the gel forming polymer and the chaotropic agent is pressed into a liquid-jet-head where the jet is splintered into droplets by the flow of a water-immiscible solvent. The droplets are caused to fall down into a column containing the same solvent but cooled to a temperature below 0° C., e.g. from −10° C. to −30° C. The droplets freeze when sedimenting along the column and are harvested in a collector. The final product in the form of beaded cryogel is obtained after thawing and rinsing with water.

The separation medium according to the present invention may also be in the form of disks or-membranes. In this case cooling of the hot solution of the gel forming polymer to the formation of a cryogel is carried out with said solution within a special form or mould. The above disks of the cryogel may be assembled to form a column-like construction in a special holder.

According to a further aspect of the invention there is provided the use of a separation medium according to the invention for the separation of cells from a cell mixture according to specific properties of their surface.

According to the invention there is also provided the use of a separation medium according to the invention which has been modified by introducing a member selected from the group consisting of ligands, charged groups and hydrophobic groups thereinto for the separation of low-molecular weight products from a cellular suspension of crude homogenate according to the charge, hydrophobicity or affinity of said products to said at least one member selected from the group consisting of ligands, charged groups and hydrophobic groups available at the separation medium.

In an embodiment of said use of the modified separation medium said medium is used for the separation of proteins from a cellular suspension or crude homogenate according to the charge, hydrophobicity or affinity of the proteins to the ligands, charged groups or hydrophobic groups available at the separation medium.

Further, the present invention provides the use of a separation medium according to the invention for the separation of viruses from a virus suspension according to specific properties of their surface.

The present invention also provides the use of a separation medium according to the invention for the separation of plasmids from crude suspensions thereof according to their surface properties, such as charge, structural organisation and base packaging.

The present invention also provides a separation method as set forth in claim 33.

The invention will now be further illustrated by means of a number of non-limitative examples.

### EXAMPLE 1

### Preparation of Supermacroporous Continuous Columns From Gel Forming Polymers in Aqueous Solution Containing Chaotropic Substance

The respective polymer as identified in the Table below was suspended in distilled water at different concentrations and heated with stirring on boiling water bath until the completion of polymer dissolution. Then the calculated amount of chaotropic agent was added and the viscous hot solution was poured slowly into a column (30×10 mm i.d.). Then the contents of these columns were frozen inside the column at different temperatures (vide Table 1 below) for 1-24 h, and thawed afterwards. The supermacroporous continuous columns thus produced were rinsed with water to wash out the soluble fractions, and the flow rate of water through these columns was measured under the hydrostatic pressure of 1 m H₂O.

The results are reported in the following Table 1.

TABLE 1
Supermacroporous continuous columns produced from the
polymer aqueous solutions containing chaotropic agents

| | Chaotropic | Polymer | | | |
|---|---|---|---|---|---|
| | agent | concen- | Freezing | Incubation in | Flow |
| | (concen- | tration | temperature, | the frozen | rate |
| Polymer | tration, M) | wt. % | ° C. | state, h | mL/h |
| Agar-agar | Urea | 2.0 | −15 | 15 | 67 |
| | (3) | | | | |
| Agar-agar | Urea | 3.0 | −20 | 24 | 90 |
| | (4) | | | | |
| Agar-agar | Acetic acid | 4.0 | −25 | 10 | 25 |
| | (1) | | | | |
| Agarose | Urea | 1.8 | −20 | 15 | 118 |
| | (3.5) | | | | |
| Agarose | Urea | 2.2 | −30 | 7.5 | 43 |
| | (4.5) | | | | |
| Agarose | NaOH | 3.0 | −10 | 18 | 124 |
| | (0.1) | | | | |
| Agarose | NaOH | 2.5 | −20 | 20 | 88* |
| | (0.1) | | | | |

*The column was composed from the porous disks of 5 mm thickness

### EXAMPLE 2

### Direct Capture of Recombinant His₆-Tagged Lactate Dehydrogenase From Particulate-Containing Crude Cell Homogenate

The continuous column prepared from agarose according to Example 1 was epoxy activated by recirculating overnight through the column a mixture of 20 ml 1,4-butanediol diglycidyl ether and 20 ml 0.6 N NaOH containing 40 mg sodium borohydride at a flow rate 2 ml/min. The column was extensively washed with water to remove excess reagent. A solution containing 2.5 g iminodiacetic acid (IDA) in 20 ml 2 M potassium carbonate was recirculated overnight through the column at a flow rate 0.2 ml/min. The column was washed with 1 liter 1 M NaCl followed by 1 liter distilled water. The excessive reactive groups were blocked by recirculating overnight through the column 15 ml 1 M ethanolamine solution pH 9.0 followed by washing with 1 liter 1 M NaCl and 1 liter distilled water. Finally, Cu²⁺ was bound to the IDA-modified column by passing 20 ml 5 mM CuSO₄ (dissolved in distilled water) through the column.

Recombinant Escherichia coli cells expressing lactate dehydrogenase (from the thermophile Bacillus stearothermophilus) carrying a tag of six histidine residues (His₆-LDH) were grown and induced for enzyme production. The cells were harvested by centrifugation, washed with 25 mM Tris-HCl buffer, pH 7.3 and disrupted by sonication.

The crude extract without pre-purification was applied on an IDA-modified agarose monolith column with bound Cu²⁺-ions at flow rate of 2 ml/min (75 cm/h). The column was washed with 25 mM Tris-HCl buffer, pH 7.3 and eluted with the same buffer containing 50 mM EDTA. The HiS6-LDH was nearly quantitatively captured from the crude extract with only 4% of the total eluted enzyme activity in the breakthrough fraction, which could be due to the admixtures of the inherent non-recombinant (and hence which cannot bind to the monolith column) lactate dehydrogenase. Bound enzyme was eluted with 83% recovery in a small volume of 50 mM EDTA of about 2 column volumes. The purification fold was 1.9.

### EXAMPLE 3

### Direct Capture of Secondary Alcohol Dehydrogenase From Particulate-Containing Crude Cell Homogenate

Affinity ligand, Procion Scarlet H-2G was immobilized on the continuous column prepared from agarose according to Example 1 by recirculating 4 M NaCl solution containing 0.1 M NaOH and 1 g/l Procion Scarlet H-2G through the column for 72 h at a flow rate of 0.2 ml/min. The column was washed finally with 1 liter 1 M NaCl followed by 1 liter distilled water.

The obligate anaerobic thermophilic organism Thermoanaerobium Brockii was cultured in batch according to J. G. Zeuss, P. W. Hegge and M. A. Andersson (1979) Arch. Microbiol. 122:41. The cells were harvested by centrifugation, washed with 20 mM morpholinopropanesulphonate buffer, pH 6.5 containing 30 mM NaCl and 2 mM MgCl₂ (MES buffer)and disrupted by sonication.

The crude extract without pre-purification was applied on an agarose monolith column with bound Procion Scarlet H-2G at a flow rate of 2 ml/min (75 cm/h). Secondary alcohol dehydrogenase was nearly quantitatively captured from the crude extract. The column was washed with MES buffer. Bound enzyme was eluted with 67% recovery in 4 column volumes of 0.5 mM NADP in MES buffer. The purification fold was 8.4.

### EXAMPLE 4

### Macroporous Agarose Beads With Fillers for Expanded Bed Chromatography

Beaded agarose cryogel was prepared using a cryogranulating set-up. Aqueous 2% (wt.) agarose solution at +65° C. was adjusted with concentrated (10 M) NaOH solution till 0.08M of alkali concentration, and then the alkali-resistant filler was dispersed in the viscous polymer solution. To increase the density of the beads to be prepared, fillers like titanium dioxide (TiO₂, density 4.2 g/cm³), zirconium dioxide (ZrO₂, density 3.8 g/cm³), molybdenum powder (Mo, density 10.2 g/cm³) or tungsten powder (W, density 19.32 g/cm³) were used. The suspension thus prepared was pressed into a liquid-jet-head where the jet was splintered into droplets by the flow of a water immiscible solvent. Droplets adopt a spherical form due to the surface tension and fall down into the column with the same solvent (e.g. petrol ether), but cooled to temperatures from −10 to −30° C. The droplets froze when sedimenting along the column and were harvested in the collector. The frozen granules were kept frozen for a certain period to form a gel and then thawed and subsequently washed with water. The diameter of the beaded filled agarose cryogel was about of 60-600 μm. The gel matrix is highly macroporous with 1-40 μm pores. The beads of filled agarose cryogel have different sizes allowing them to form a stable expanded bed when a mobile phase is pumped from beneath the column, with smaller particles accumulating in the upper part and larger particles accumulating in the lower part of the of the expanded bed.

Bed expansion studies for these cryogels has been carried out in 1.0 cm i.d. column with movable adapters at both ends and a flow distributor consisting of teflon disc with 8 holes of 0.5 mm diameter at the base of column using deionized water or 50 mM Na-phosphate buffer, pH 7.0 at different linear velocities. The settled bed height at the start of the experiments was 3-6 cm. The expanded bed column was connected to a peristaltic pump (Labchem, Sweden). After each change in the flow rate, 10 min were allowed for the bed to stabilize. Expanded bed height was measured as a function of the liquid linear velocity. Beaded filled agarose cryogel gave a stable expansion.

1-14. (canceled)

15. A method for the preparation of a separation medium in macroporous gel form by cooling an aqueous solution of at least one gel forming polymer to a temperature, at which the solvent in the system is partially frozen with the dissolved substances concentrated in the non-frozen fraction of the solvent, characterized in that said gel forming polymer is selected from the group consisting of polymers normally forming gels too fast when an aqueous solution thereof is cooled to a temperature within a range below 0° C. to enable the formation of a cryogel and that said cooling is carried out in the presence of at least one chaotropic agent in said aqueous solution in order to prevent gel formation before the polymer solution is frozen.

16. A method according to claim 15, wherein said at least one polymer is a polysaccharide selected from the group consisting of agarose, agar, carrageenans, starch and cellulose and their respective derivates or a mixture of said polysaccharides.

17. A method according to claim 15, wherein said at least one chaotropic agent is selected from the group consisting of urea, alkyl ureas, guanidine chloride, LiCl, KSCN, NaSCN, acids and bases and mixtures thereof.

18. A method according to claim 15, wherein the separation medium thus prepared is subsequently cross-linked.

19. A method according to claim 18, wherein cross-linking is carried out by means of a cross-linking agent selected from the group consisting of epichlorohydrin, divinyl sulfone, glutaric dialdehyde, azidobenzoyl hydrazide,4-(N-maleimido-methyl)cyclohexane-1-carboxyl hydrazide hydrochloride, N-hydroxysuccinimidyl-4-azidosalicylic acid, 3-(2-pyridyldithio)-propionyl hydrazide, dimethyladipimidate*2HCL, N-succinimidyl-6(4′-azido-2′-nitrophenylamino) hexanoate and sulfosuccinimidyl-(4′-azidosalicylamido) hexanoate, di- and triglycidyl compounds.

20. A method according to claim 15, wherein the separation medium thus prepared is modified by introducing a member selected from the group consisting of ligands, charged groups and hydrophobic groups thereinto.

21. A method according to claim 20, wherein said modification is carried out by introducing a member selected from the group consisting of dyes and ion exchange groups into said separation medium.

22. A method according to claim 15, wherein cooling of said aqueous solution of gel forming polymer (s) and chaotropic agent (s) is carried out in the presence in said solution of a filler in order to increase the density of the separation medium or to introduce a ligand thereinto.

23. A method according to claim 22, wherein the filler is selected from the group consisting of metals, metal oxides and ion exchange substances in the form of particles.

24. A method according to claim 15 , wherein the separation medium is prepared in the form of a monolith en-cased in a column.

25. A method according to claim 15, wherein the separation medium is prepared in the form of particles.

26-30. (canceled)

31. Method for the separation of
a) cells from a cell mixture according to specific properties of their surface;
b) low-molecular weight products or proteins from a cellular suspension or crude homogenate according to charge, hydrophobicity or affinity of said products to at least one member selected from the group consisting of ligands, charged groups and hydrophobic groups;
c) viruses from a virus suspension according to specific properties of their surface; or
d) plasmids from crude suspensions thereof according to their surface properties, by contacting said cell mixture, cellular suspension or crude homogenate, virus suspension and crude plasmid suspension with a separation medium for adsorption of cells, low- molecular weight products or proteins, viruses and plasmids, respectively, to said separation medium and then eluting them therefrom.
